(19) United States
(12) Patent Application Publication
(10) Pub. No.: US 2002/0002354 A1
(43) Pub. Date: Jan. 3, 2002

(54) Syringe for medical purposes

(75) Inventors: Udo J. Vetter, Ravensburg (DE); Joachim Glocker, Weingarten (DE); Thomas Otto, Weingarten (DE); Eugen Frasch, Oberteuringen (DE)

Correspondence Address: THE FIRM OF KARL F ROSS, 5676 RIVERDALE AVENUE, PO BOX 900, RIVERDALE (BRONX), NY, 10471-0900, US

(73) Assignee: Arzneimittel GmbH Apotheker Vetter & Co. Ravensburg

(21) Appl. No.: 09/835,291

(22) Filed: Apr. 13, 2001

(30) Foreign Application Priority Data
Apr. 15, 2000 (DE) 10018827.3

(51) Int. Cl. A61M 5/178; A61M 5/32

(52) U.S. Cl. 604/272; 604/164.11

(57) ABSTRACT

A syringe has a protective sleeve with finger seats effective in both axial directions and the thumb rest of the plunger is also effective in both axial locations, so that, after injection, the fingers and thumb are spread apart to advance the protective sleeve over the needle.

### FIELD OF THE INVENTION

Our present invention relates to a syringe for medical purposes, more particularly, to a syringe which has a protective sleeve which can be displaced into a position in which it surrounds the needle or cannula and thereby prevents undesired pricking by such needle or cannula

### BACKGROUND OF THE INVENTION

Syringes of the type which have a protective sleeve which can be displaced over the needle or cannula are described, for example in EP 0740942.

Such a syringe, used for medicinal purposes can comprise a syringe cylinder or barrel which can be formed at one end with a fitting receiving a fixed cannula or needle or forming a connector for a detachable cannula or onto which a cannula may be mounted by an appropriate attachment part. The syringe has a shiftable plug which can be displaced by a plunger rod (piston rod) by the thumb of the user and a protective sleeve which surrounds the syringe cylinder and prior to and/or after use can be displaced into a position in which the cannula at least in the region of its type is received within the sleeve. The sleeve can be arrested in this position and for that purpose may have a detent groove along is inner surface into which an annular shoulder surrounding the fitting can engage.

In the syringe of EP 0 740 942, the sleeve primarily serves to protect medical personnel and the patient from coming into contact with the pointed end of the cannula unintentionally. This is especially important to prevent the spread of blood-borne infectious diseases.

Usually the entire injection process is effected with one hand so that the second hand remains free, for example, to swipe a swab over the injection site. With the earlier syringe it is not possible to then shift the protective sleeve over the needle since the second hand is required for that purpose and remains engaged elsewhere. As a result a certain risk of inadvertent pricking by the cannula remains.

### OBJECTS OF THE INVENTION

It is, therefore, the principal object of the present invention to provide a syringe of the type described here so that even the securing of the cannula, i.e. the shifting of the sleeve over the cannula, can be effected by the one hand involved in the injection process.

Another object of the invention is to provide a medicinal syringe which eliminates drawbacks of earlier syringes.

It is also an object of the invention to provide a syringe for medicinal purposes which affords greater security against the spread of blood-borne diseases than earlier syringes.

### SUMMARY OF THE INVENTION

These objects and others which will become apparent hereinafter are attained, in accordance with the invention, by providing on the protective sleeve in the syringe of the type described, a finger receiver which preferably can engage the index and middle fingers of the user, i.e. the two fingers usually used to hold the syringe barrel, but configured so that the fingers are braced against the sleeve in both axial directions, i.e. toward and away from the cannula. According to the invention also, the plunger or piston rod has at its free end an engagement element for the thumb which is likewise configured so that it can be displaced by the thumb in both axial directions, i.e. toward and away from the cannula. The advantage which results from this construction is that, subsequent to a complete depression of the plunger into the injection cylinder, the user can again spread his or her thumb and fingers to shift the protective sleeve over the cannula until its detent engages and it is locked in the position in which contact with the needle is permanently prevented.

The engagement of the thumb on the plunger can be assured in an especially simple manner by forming the plunger with a thumb seat which overhangs the plunger shank and thus permits the thumb to engage below this seat. The thumb normally bears upon the seat to drive that plunger into the syringe cylinder and, once the plunger is in its fully retracted position, the thumb of the user can engage below or behind this seat, usually a flange injection molded on the plunger, for the spreading of the thumb and fingers which shifts the sleeve over the cannula.

It is also possible to shape the thumb rest differently and in that respect the thumb engaging element can be a partly or fully closed ring.

To the extent that the protective sleeve may be provided wo that it can easily slide over the injection cylinder and the plunger may have a sufficiently high degree of sliding friction within the cylinder to prevent the plunger form being pulled out during the spreading of the thumb and fingers to displace the protective sleeve, no additional elements are required to hold the plunger in its fully retracted position within the syringe cylinder. However, if there is a possibility that the pressure of the thumb on the underside of the thumb seat or tending to move the plunger away form the needle end of the syringe, we can provide a detent device at the end of the plunger proximal to the thumb seat which will retain the plunger in its fully inserted position and thus against the plug which in turn can bear against a stop within the syringe cylinder. This detent at the end of the plunger remote from the plug can cooperate with a formation on the syringe cylinder, likewise at the end thereof remote from the cannula.

More specifically, the detent can be formed by a flange member which can clip onto the outwardly extending head at the open end of the syringe cylinder remote from the fitting. The flange member can be provided with a bore coaxial with the cylinder and through which the plunger passes, the detent formation being provided on the edge of the bore or as an edge of the bore engageable in a toothing on the plunger or piston rod.

The detent can include a sleeve on an edge of the bore and an annular projection on the plunger in a kinematic reversal of the first-mentioned detent construction.

To ensure an effective retention of the plunger, it has been found to be advantageous to provide the toothing on the plunger as a plurality of axially offset teeth, i.e. a row of teeth. It has also been found to be advantageous to provide the finger receiver as a pair of spaced-apart annular members. One of these annular members can be a radially projecting collar at the end of the protective sleeve remote from the cannula end. The other of these members may be a radially outwardly projecting bulge. The receiver as thus constituted has been thus found to provide secure engagement by the fingers both during injection and the subsequent shifting of the protective sleeve.

So that the protective sleeve can slide easily upon the injection cylinder it has been found to be advantageous to form the protective cylinder on its inner surface with two radially inwardly directed annular projections which ride upon the outer surface of the sleeve, one of which is provided in the region of the collar at the respective end of the protective sleeve while the other is formed substantially midway along the protective sleeve and is formed with the detent groove.

It is also possible to provide the protective sleeve, at its end turned toward the cannula, with a removable safety cap which can be inspected to ensure that the syringe has not been tampered with and still is in its original packaged condition prior to injection. After removal of this safety cap, the injection can be prepared it is also possible within this concept that the safety cap have an elastic cannula-protecting cap received therein.

### BRIEF DESCRIPTION OF THE DRAWING

The above and other objects, features, and advantages will become more readily apparent from the following description, reference being made to the accompanying drawing in which:

FIG. 1a is an axial section of a syringe according to the invention illustrating the removal of the protective cap;

FIG. 1b is a view similar to FIG. 1a showing a further step in the removal of the protective cap;

FIG. 1c shows the injection state of the syringe;

FIG. 1d is a similar view showing the position of the parts at the end of the injection and prior to spreading of the fingers and thumb to displace the protective sleeves;

FIG. 1e is a similar view showing the displacement of the protective sleeve;

FIG. 1f shows the protective sleeve permanently anchored around the cannula;

FIG. 1g is a detail of the region Ig of FIG. 1e;

FIG. 1h is a detail of the region Ih of FIG. 1f;

FIGS. 2a-2f are assembly diagrams partly in cross section and partly in elevation illustrating the assembly of the device;

FIG. 3a is a diagram showing the final injection position equivalent to FIG. 1d in the prior sequence.

FIG. 3b is a detail of the region IIIb of FIG. 3a;

FIG. 4a shows the beginning of the spreading operation;

FIG. 4b and 4c show other configurations of the thumb engaging member;

FIG. 5a is another illustration of the protected syringe;

FIG. 5b is a detail of the region Vb of another embodiment of the invention; and

FIGS. 5c and 5d are views similar to FIG. 5b but illustrating other embodiments.

### SPECIFIC DESCRIPTION

The syringe shown in the drawing are used for injection and like medicinal purposes and can comprise a syringe cylinder 1, one end of which is provided with a fixed needle 2 or another fitting 3 for receiving a removable needle or a needle equipped with a tapered sleeve fitting over that fitting. A closure member such as a Tip-cap within the syringe cylinder 1, a shiftable plug 4 is disposed and this shiftable plug can be actuated by the plunger 5. The syringe cylinder 1 is surrounded by a protective sleeve 6 which, following the medical use of the syringe, can be displaced until it surrounds and prevents contact with the cannula 2 up to and including its point. In this position the protective sleeve 6 can be arrested and for this plug and for this pumps 3a be inwardly open detent groove 7 which, when aligned with an annular shoulder 8 can receive the latter and lock the sleeve in place. The flanks leading to the groove are bevelled to enable the shoulder 8 to jump into the groove. The shoulder 8 is provided around the fitting 3.

The protective sleeve 6 is formed on its outer surface with a receiver or seat 9 adapted to receive the two fingers of the user which usually engage the syringe body during an injection. The seat 9 can be engaged by the fingers in both axial directions, thus for displacement of the sleeve toward the cannula and for drawing the sleeve in a direction away from the cannula.

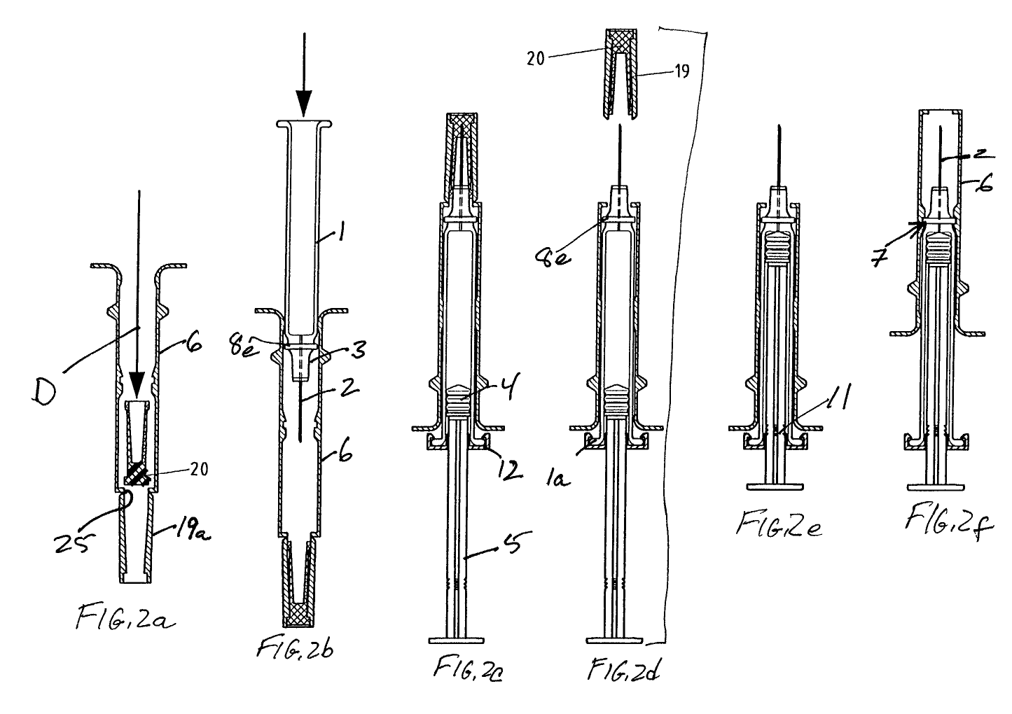

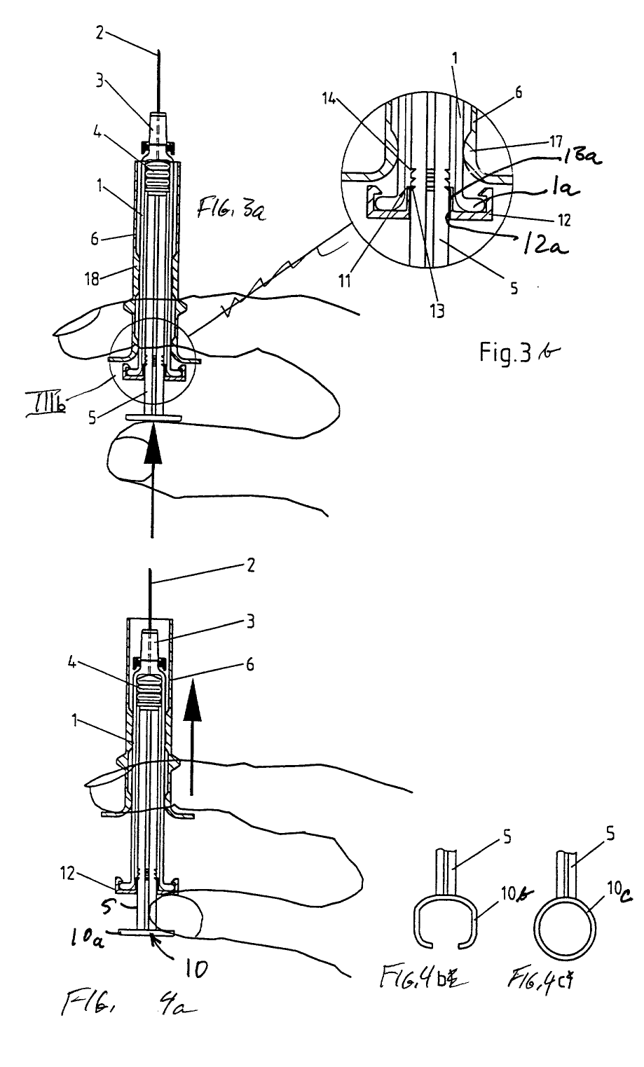

In addition, the plunger 5 is provided at its end opposite the plug 4 with a thumb rest 10 which can also be engaged by the thumb in two opposite axial directions. As a consequence, it is possible, as can be seen especially from a comparison of FIGS. 3a and 4a to effect the injection conventionally, whereby the index finger and the middle finger hold the sleeve and hence the cylinder while the thumb presses the plunger toward the needle and then spread the thumb away from the fingers to shift the protective sleeve 6 over the needle in the direction of the area in FIG. 4a. In this case the thumb engages behind the thumb rest 10.

The thumb rest 10 can be a plate 10a formed unitarily on the plunger 5 (FIG. 4a) or a loop 10b or even a ring 10c as can be seen from FIGS. 4b and 4c. The thumb rest 10b can be referred to as an open ring or a ring having a gap.

To prevent the plunger 5 from being inadvertently withdrawn from the cylinder during the spreading movement and advance to the sleeve 6 over the needle, a detent 11 is provided to hold the plunger 5 in its fully retracted position. This detent 11 is formed as a flange member 12 (FIG. 3b) which can be clipped over the outwardly extending bead 1a of the syringe body and can have an opening or bore 12a through which the plunger 5 passes along this bore is a sleeve 13a formed with inward projections 13 which can engage in rows of teeth 14 provided on the plunger 5 close to the thumb rest 10.

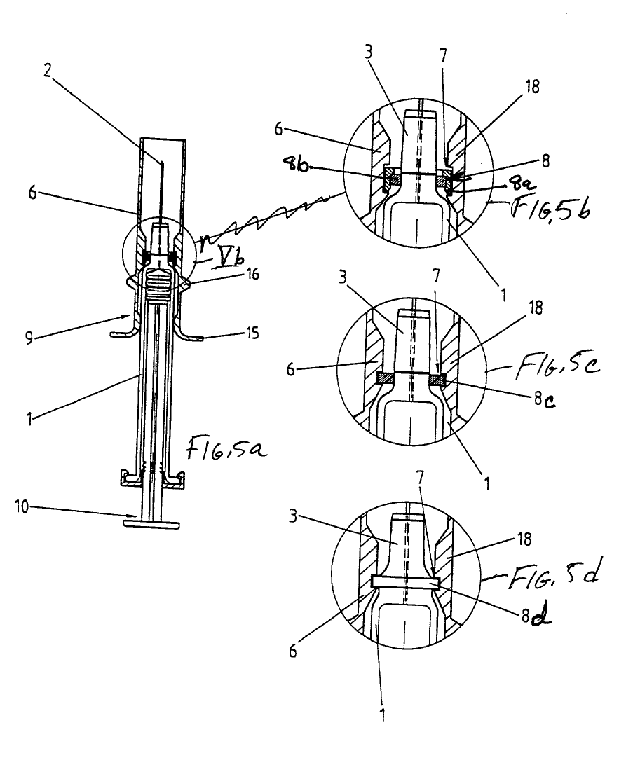

The finger seat 9, which usually is engaged by the index and middle fingers can comprise, as shown in greater detail in FIG. 5a, a radially projecting collar 15 at the end of the sleeve 6 and, axially spaced therefrom, an outwardly projecting annular bead 16.

In addition the protective sleeve 6 is formed along its interior with two radially projecting annular formations 17 and 18 with which the sleeve 6 rides on the cylinder 1. One of these annular projections 17 is located in the region of the collar 15 at the end of the sleeve 6 while the other projection 18 is located generally midway along the sleeve 6 and is formed with the detent groove 7 previously mentioned.

The protective sleeve 6 can be provided, on its end turned toward the cannula 2 with a separable but connected safety cap which can ensure that the syringe is in its original state prior to injection. The safety cap can be broken off from the sleeve 6 or the shoulder 8 mentioned previously. Within the safety cap 19, an additional elastic needle protective cap 20 can be mounted and in which the needle can be received. After separation of the safety cap 19, with which the needle protective cap 20 is removed, the syringe, following the usual venting, is appropriate for injection.

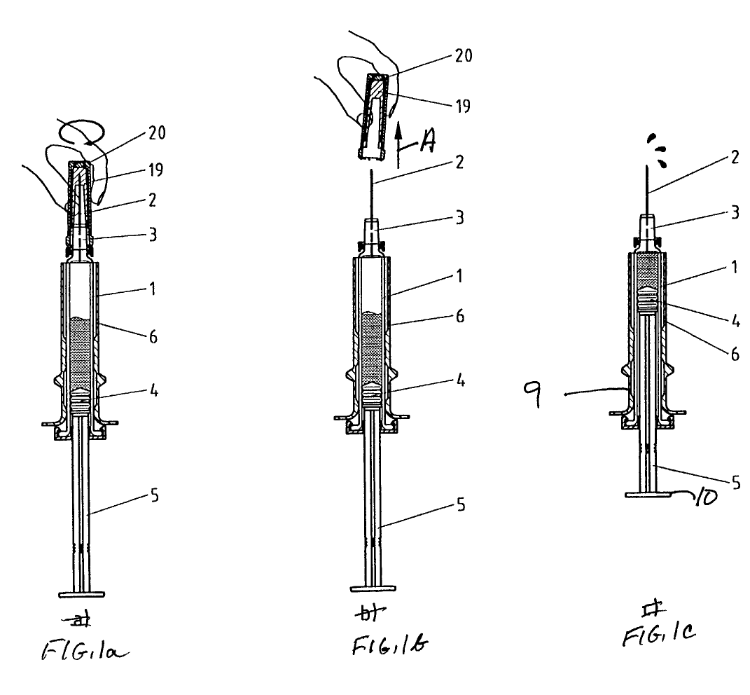

Turning to FIGS. 1a through 1h, therefore, it will be apparent that, with the syringe in its original preinjection position shown in FIG. 1a, the cap 19, 20 can be removed (Arrow A in FIG. 1b) and the syringe vented (FIG. 1c) by advancing the plunger 5. This is achieved by engaging the index finger and the middle finger in the seat 9 and pressing with the thumb on the thumb rest 10. The pressing action continues (arrow B in FIG. 1d) until the plunger 5 and the plug 4 have driven the contents of the syringe through the needle 2 into the patient.

At that point, the detent 11 engages (FIG. 1e) and the thumb is shifted behind the thumb rest 10 and spread away from the fingers to thereby displace the sleeve 6 in the direction of arrow C until the shoulder 8 snaps into the groove 7 (FIG. 1f and 1h).

The shoulder 8 can be formed by a plastic ring 8a fitted onto an elastic member 8b, can be an elastic member 8c itself (FIGS. 5b and 5c) or can be molded from the material of the syringe body as shown for the annular shoulder 8d in FIG. 5d.

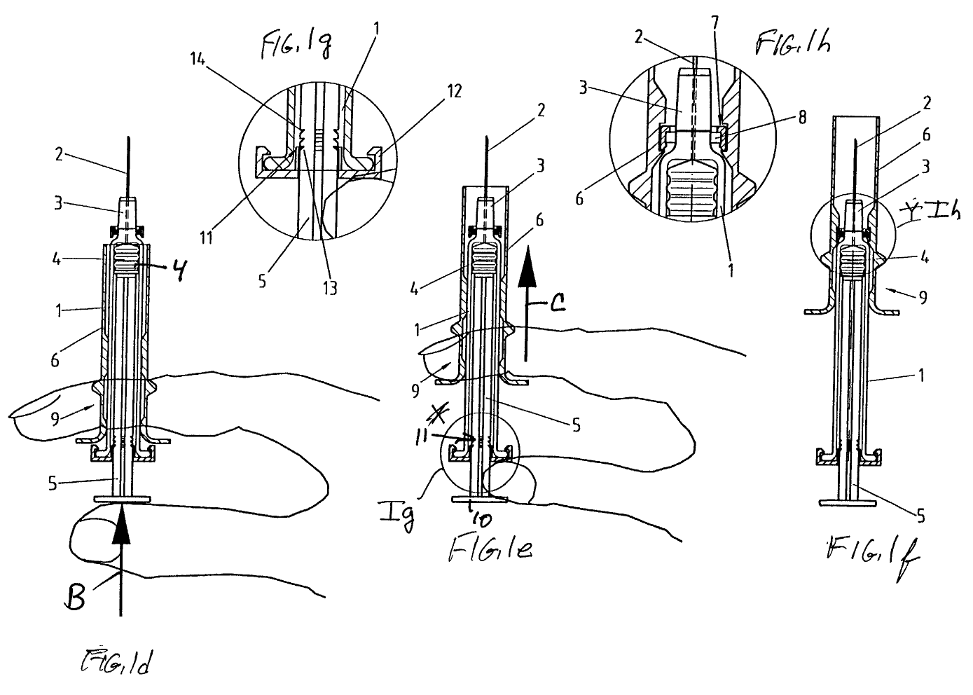

FIGS. 2a - 2f show the assembly of the syringe. As can be seen from FIG. 2a, a sleeve 19a forming the cap 19 is injection molded as part of the sleeve 6 with a break-away thin zone at 25. The member 20 which can be composed of foam rubber is inserted into the sleeve 19a into the direction of the arrow D.

The syringe cylinder 1 is then inserted into the sleeve 6 and in the embodiment shown in FIG. 2b has the needle 2 permanently attached to the fitting 3 and the latter has an annular collar 8e formed thereon from the material of the syringe :; body, usually glass.

The syringe can then be filled and the flange member 12 snapped onto the bend 1a and the plug 4 and the plunger 5 can then be applied. The needle 2 is ensconced in the cap. From FIGS. 2d - 2f, the sequence of the invention is followed, namely, the cap 19, 20 is removed and the thumb wheel displaces the plunger 5 to employ the syringe. The detent 11 is engaged and the sleeve 6 advanced until the detent 7 is effective to lock the sleeve 6 over the needle 2.

We claim:

1. A syringe for medicinal purposes comprising a syringe cylinder formed at one end with a fitting adapted to receive a cannula;
a plug shiftable in said cylinder toward said end to displace contents of said cylinder through said cannula;
a plunger engaging said plug and extending out of an opposite end of said cylinder said plunger being formed with a thumb rest engageable by said thumb in two opposite axial directions; and
a protective sleeve surrounding said cylinder and slidable therealong, said sleeve being formed with an inwardly open detent groove and said formation having a detent member engageable in said groove upon displacement of said sleeve over said cannula, said protective sleeve being formed with a receiver for fingers of the user axially engageable by the fingers in opposite axial directions whereby, upon spreading of the thumb from the fingers, said sleeve is displaced along said cylinder to engage said detent member in said groove.

2. The syringe defined in claim 1 wherein said thumb rest is a radially outwardly extending plate on which and behind which said thumb is engageable.

3. The syringe defined in claim 1 wherein said thumb rest is formed as an at least partially closed ring.

4. The syringe defined in claim 1, further comprising means at said other end of said cylinder and on said plunger for retaining said plunger in a position in which said plunger is at a maximum degree of insertion in said cylinder.

5. The syringe defined in claim 4 wherein said means includes an inwardly extending member and at least one tooth on said plunger engageable by said inwardly extending member.

6. The syringe defined in claim 5 wherein said means includes a flange member clipped onto a bead at said other end of said cylinder and formed with said inwardly extending member, said plunger being formed with a row of teeth in which said inwardly extending member engages.

7. The syringe defined in claim 6 wherein said inwardly extending member is a sleeve along a bore of said flange member and a radial annular projection on said sleeve.

8. The syringe defined in claim 1 wherein said seat is formed by a radially outwardly extending collar on said sleeve and a radially outwardly projecting bulge axially spaced from said collar.

9. The syringe defined in claim 1 wherein said sleeve is formed with a first annular inward projection adjacent an end of said sleeve remote from said needle and a second inward projection located substantially midway along said sleeve and formed with said groove.

10. The syringe defined in claim 1, further comprising a cap separable from said cylinder and covering said cannula for evidencing tampering with the syringe.

11. The syringe defined in claim 1 wherein said cap is formed in one piece with said sleeve and adjoins said sleeve by a breakaway portion.

12. The syringe defined in claim 11, further comprising an elastic cap portion within said cap for engagement with said cannula.
